(19) United States
(12) Patent Application Publication
(10) Pub. No.: US 2009/0163540 A1
(43) Pub. Date: Jun. 25, 2009

(54) Quinine Sulfate Polymorphs, Processes of Preparing, Compositions and Uses Thereof

(76) Inventors: Tong Sun, Marlton, NJ (US); Keith Lorimer, West Lafayette, IN (US); Kevin Wayne Meyer, Lebanon, IN (US); Kurt R. Nielsen, Chadds Ford, PA (US); Shawn Watson, Cherry Hill, NJ (US)

Correspondence Address: CANTOR COLBURN, LLP, 20 Church Street, 22nd Floor, Hartford, CT, 06103, US

(21) Appl. No.: 12/336,811

(22) Filed: Dec. 17, 2008

Related U.S. Application Data
(60) Provisional application No. 61/015,488, filed on Dec. 20, 2007.

(51) Int. Cl. A61K 31/439; C07D 401/06; A61P 33/06

(52) U.S. Cl. 514/305; 546/134

(57) ABSTRACT

Disclosed are new quinine sulfate polymorphs, methods of making the polymorphs as well as formulations prepared therefrom and uses thereof.

### CROSS REFERENCE TO RELATED APPLICATION

This application claims the benefit of U.S. Provisional Application Ser. No. 61/015,488 filed Dec. 20, 2007, which is hereby incorporated by reference in its entirety.

### BACKGROUND

Quinine (cinchonan-9-ol, 6′-methoxy-, (8α,9R)-) is an antiprotozoal and an antimyotonic, and is known for the treatment of malaria caused by Plasmodium species, the treatment of nocturnal recumbency leg muscle cramps, and the treatment of babesiosis caused by Babesia microti.

Various salts of quinine are known including quinine hydrochloride and quinine sulfate.

Polymorphs are solid crystalline phases of an active agent differing by the arrangement of the active agent molecules in the solid state. Different polymorphs of the same active agent can exhibit different physical properties such as solubility, melting point, hardness, optical properties, dissolution, and the like. Differences in the dissolution of the polymorphs can result in differences in the therapeutic activity between the different polymorphs.

Polymorphism is an important consideration in formulating an active agent, specifically in regard to the stability of active agent. Use of a metastable polymorph may lead to conversion to a stable polymorph, resulting in variable quality of the dosage form, batch to batch.

There remains a need in the art for new forms of quinine having improved properties of solubility, stability, processability and the like.

### SUMMARY

In one embodiment, disclosed herein are quinine sulfate polymorphs B, C, D, E, F, G, H, I, J, and K and methods of making thereof. The quinine sulfate polymorphs are useful in the treatment of malaria caused by Plasmodium species, uncomplicated Plasmodium falciparum malaria, severe or complicated Plasmodium falciparum malaria, malaria caused by Plasmodium vivax, leg muscle cramps, or babesiosis; or for the prophylaxis of malaria or leg muscle cramps.

These and other embodiments, advantages and features of the present invention become clear when detailed description and examples are provided in subsequent sections.

### BRIEF DESCRIPTION OF DRAWINGS

FIG. 1 illustrates an XRPD pattern of quinine sulfate Form B.

FIG. 2 illustrates an XRPD pattern of quinine sulfate Form C.

FIG. 3 illustrates an XRPD pattern of quinine sulfate Form D.

FIG. 4 illustrates an XRPD pattern of quinine sulfate Form E.

FIG. 5 illustrates an XRPD pattern of quinine sulfate Form F.

FIG. 6 an XRPD pattern of quinine sulfate Form G.

FIG. 7 illustrates an XRPD pattern of quinine sulfate Form H.

FIG. 8 illustrates an XRPD pattern of quinine sulfate Form I.

FIG. 9 illustrates an XRPD pattern of quinine sulfate Form J.

FIG. 10 illustrates an XRPD pattern of quinine sulfate Form K.

FIG. 11 illustrates an XRPD pattern of quinine sulfate Form A.

### DETAILED DESCRIPTION

It has been unexpectedly discovered herein that quinine sulfate exists in several polymorphs as an anhydrate, dihydrate, and solvate. Known crystalline quinine sulfate dihydrate is designated as Form A. Generated solids with unique powder patterns are designated Forms B-K. Forms B-K may be hydrated or solvated. Forms B-K may be differentiated of the basis of their XRPD peaks exemplified, for example, in Tables 1-10 below. Polymorphs may also be differentiated by their melting point as determined by, for example, differential scanning calorimetry; by their FT-IR spectra; and by their crystalline unit cell parameters. Also included are methods of making the quinine sulfate polymorphs.

In one embodiment, a quinine sulfate polymorph is substantially free of other quinine sulfate polymorphs. As used herein, substantially free means comprising less than 2 wt % of other quinine sulfate polymorphs.

In another embodiment, a quinine sulfate polymorph has a purity of greater than or equal to about 95, 96, 97, 98, 99 or 99.5%.

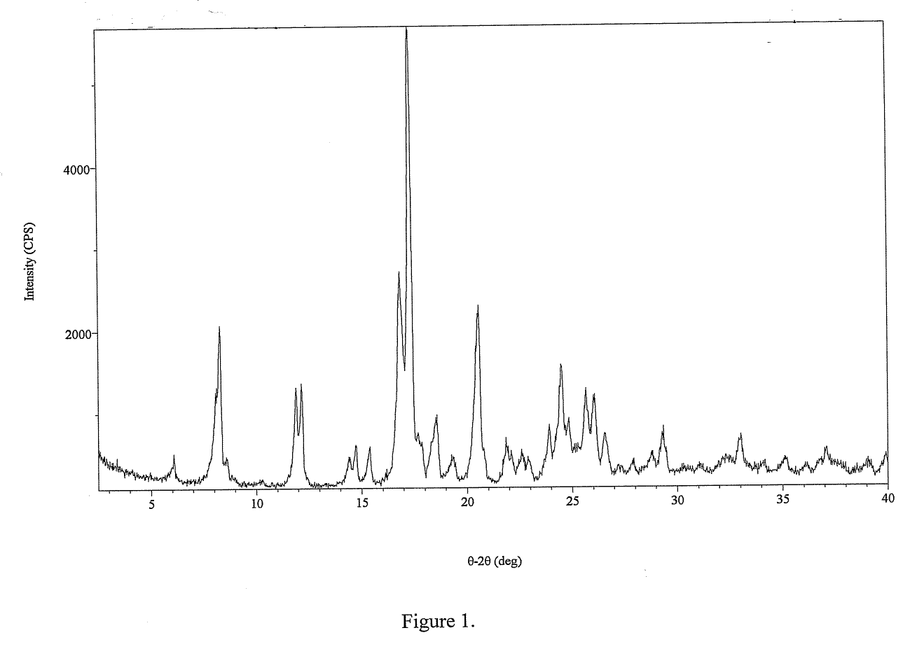

In one embodiment, quinine sulfate polymorph Form B exhibits XRPD peak positions at 8.3, 11.9, 12.2, 16.8, 17.3, 18.5, 20.6, 24.5, 25.7, and 26.1 ±0.2 degrees 2-theta. In another embodiment, quinine sulfate polymorph Form B exhibits the XRPD peak positions in Table 1. In yet another embodiment, quinine sulfate polymorph Form B exhibits an X-ray powder diffraction pattern which is substantially similar to FIG. 1.

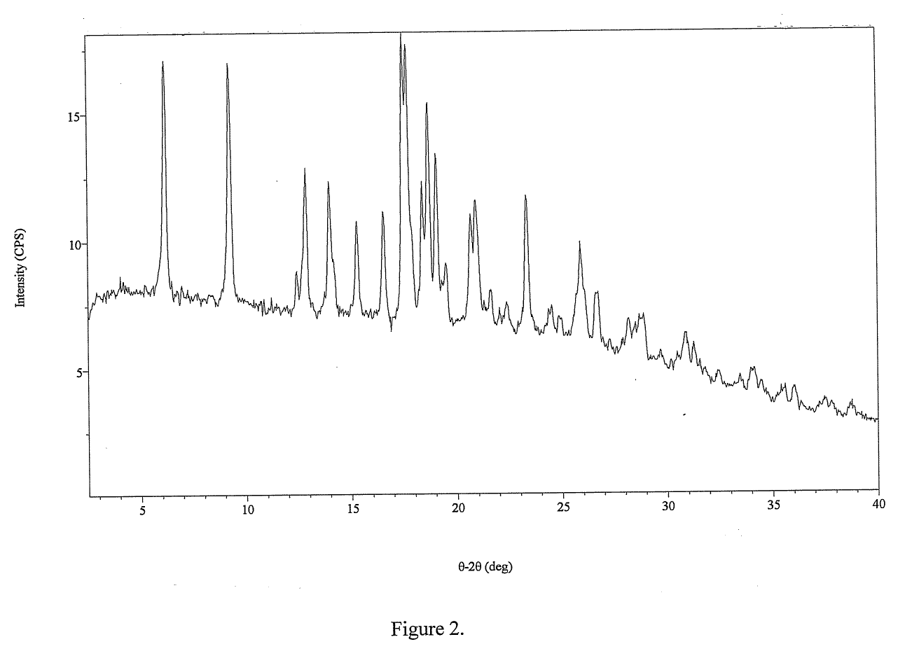

In one embodiment, quinine sulfate polymorph Form C exhibits XRPD peak positions at 6.2, 9.2, 12.9, 14.0. 15.3, 16.6, 17.5, and 18.4 ±0.2 degrees 2-theta. In another embodiment, quinine sulfate polymorph Form C exhibits the XRPD peak positions in Table 2. In yet another embodiment, quinine sulfate polymorph Form C exhibits an X-ray powder diffraction pattern which is substantially similar to FIG. 2.

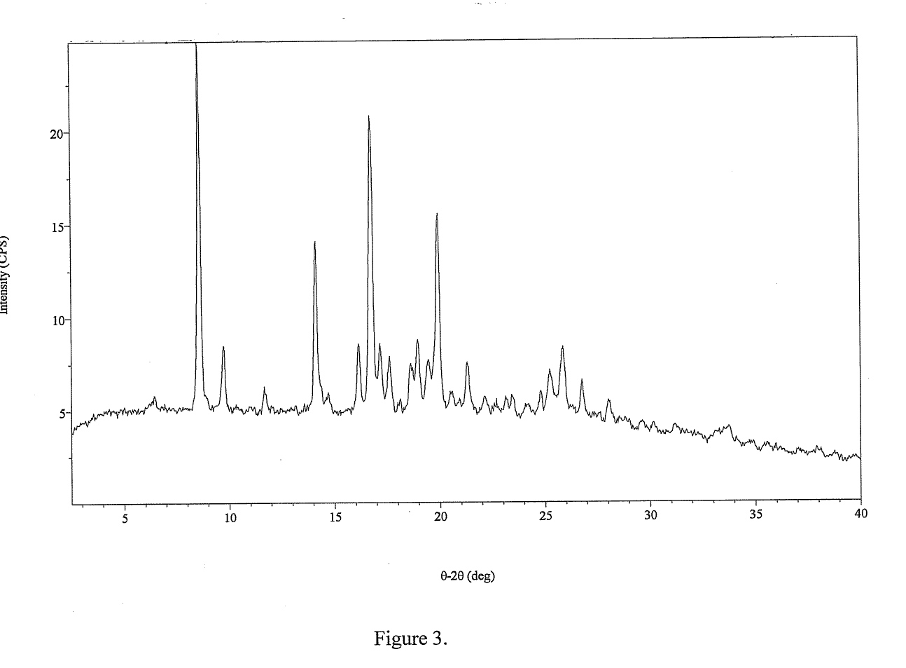

In one embodiment, quinine sulfate polymorph Form D exhibits XRPD peak positions at 8.6, 9.7, 14.1, 16.8, 18.1, 19.9, and 21.3 ±0.2 degrees 2-theta. In another embodiment, quinine sulfate polymorph Form D exhibits the XRPD peak positions in Table 3. In yet another embodiment, quinine sulfate polymorph Form D exhibits an X-ray powder diffraction pattern which is substantially similar to FIG. 3.

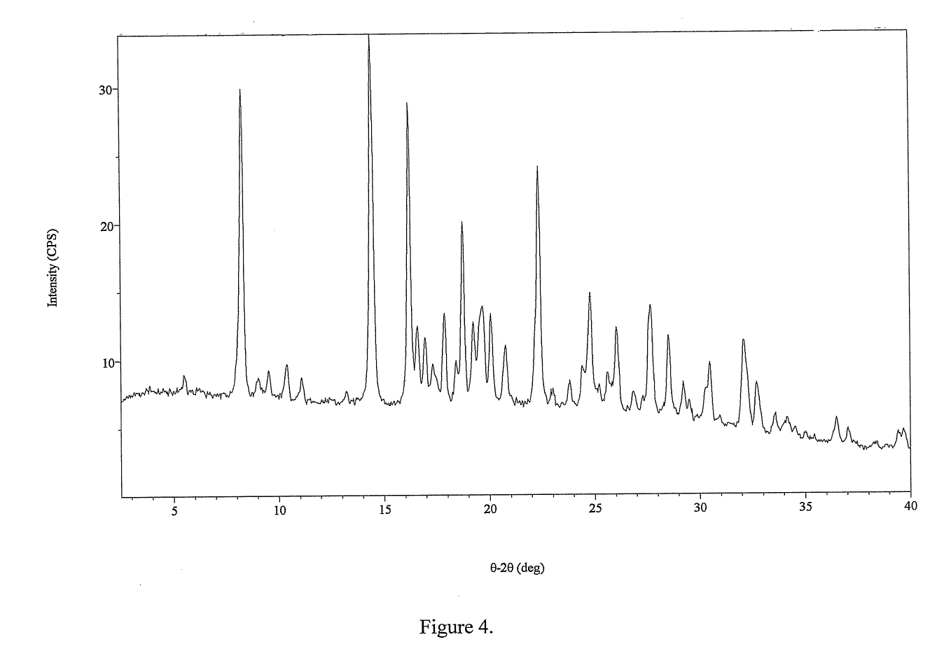

In one embodiment, quinine sulfate polymorph Form E exhibits XRPD peak positions at 8.3, 14.4, 16.2, 17.9, 18.8, 22.4 and 26.0 ±0.2 degrees 2-theta. In another embodiment, quinine sulfate polymorph Form E exhibits the XRPD peak positions in Table 4. In yet another embodiment, quinine sulfate polymorph Form E exhibits an X-ray powder diffraction pattern which is substantially similar to FIG. 4.

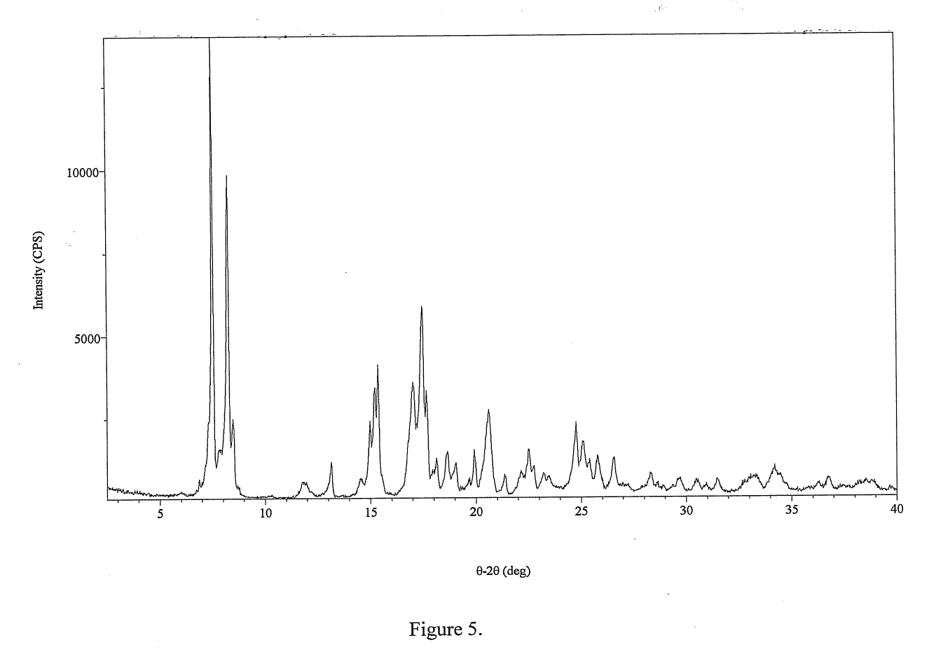

In one embodiment, quinine sulfate polymorph Form F exhibits XRPD peak positions at 7.5, 8.3, 15.4, 17.5, and 20.6 ±0.2 degrees 2-theta. In another embodiment, quinine sulfate polymorph Form F exhibits the XRPD peak positions in Table 5. In yet another embodiment, quinine sulfate polymorph Form F exhibits an X-ray powder diffraction pattern which is substantially similar to FIG. 5.

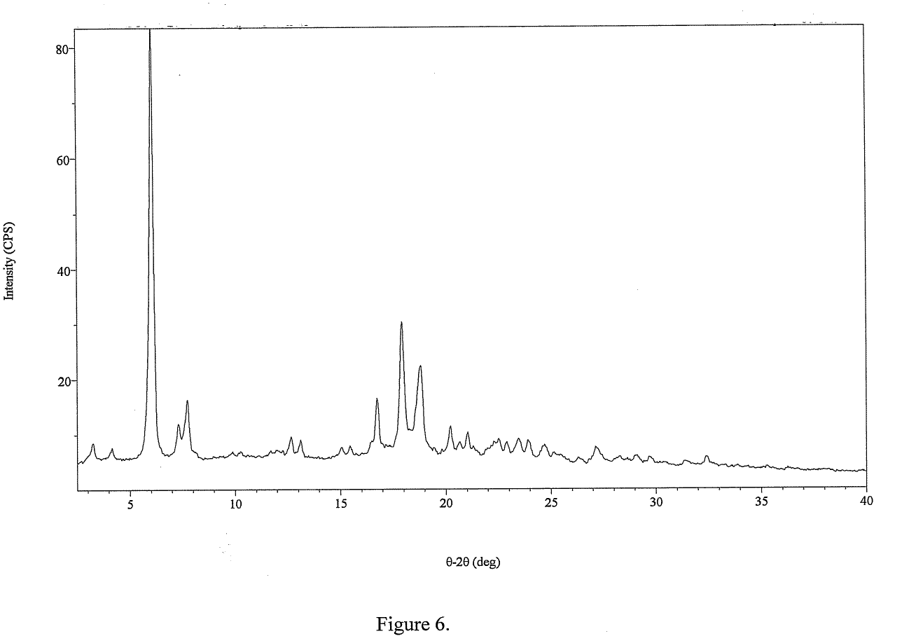

In one embodiment, quinine sulfate polymorph Form G exhibits XRPD peak positions at 6.1, 7.7, 16.8, 17.9, and 18.8 ±0.2 degrees 2-theta. In another embodiment, quinine sulfate polymorph Form G exhibits the XRPD peak positions in Table 6. In yet another embodiment, quinine sulfate polymorph Form G exhibits an X-ray powder diffraction pattern which is substantially similar to FIG. 6.

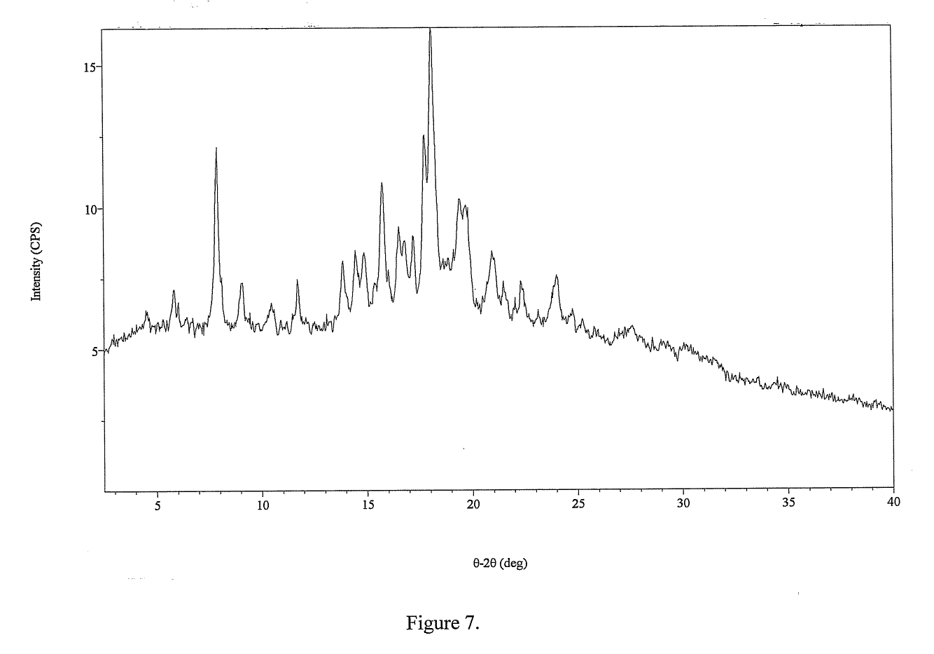

In one embodiment, quinine sulfate polymorph Form H exhibits XRPD peak positions at 7.9, 9.1, 13.9, 15.8, 16.5, 17.2, 17.8, and 18.1 ±0.2 degrees 2-theta. In another embodiment, quinine sulfate polymorph Form H exhibits the XRPD peak positions in Table 7. In yet another embodiment, quinine sulfate polymorph Form H exhibits an X-ray powder diffraction pattern which is substantially similar to FIG. 7.

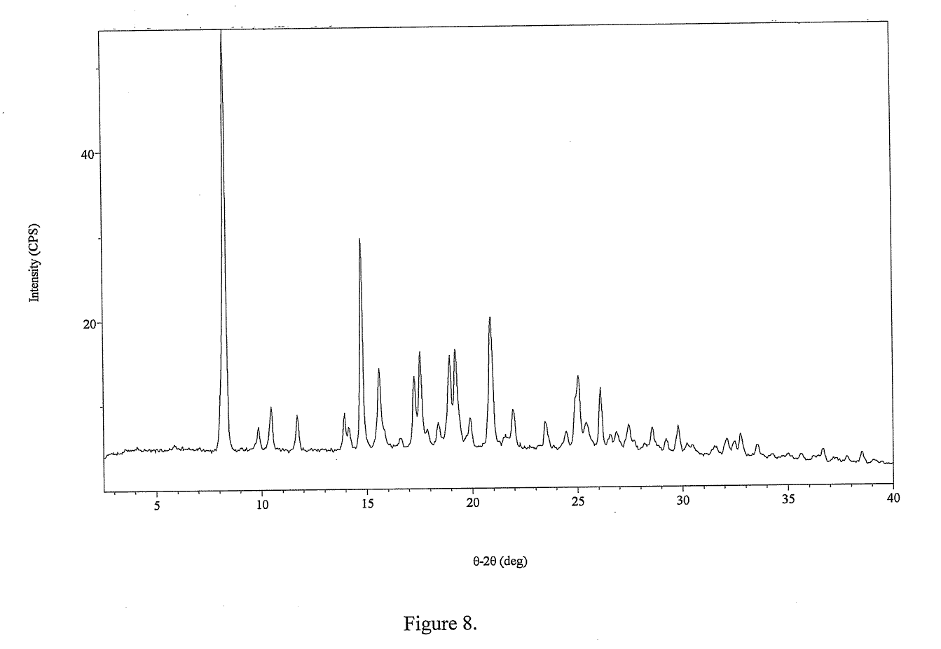

In one embodiment, quinine sulfate polymorph Form I exhibits XRPD peak positions at 8.3, 10.5, 14.8, 15.6, 17.2, 17.6, 18.9, 19.2, and 20.9 ±0.2 degrees 2-theta. In another embodiment, quinine sulfate polymorph Form I exhibits the XRPD peak positions in Table 8. In yet another embodiment, quinine sulfate polymorph Form I exhibits an X-ray powder diffraction pattern which is substantially similar to FIG. 8.

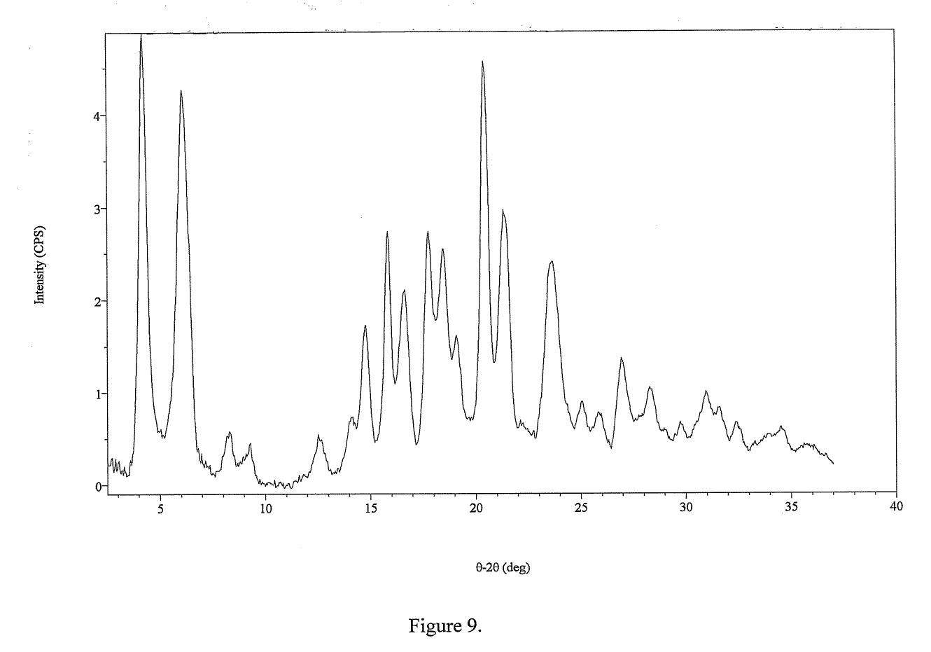

In one embodiment, quinine sulfate polymorph Form J exhibits XRPD peak positions at 4.2, 6.1, 15.8, 18.4, 20.4, 21.4, and 23.6 ±0.2 degrees 2-theta. In another embodiment, quinine sulfate polymorph Form J exhibits the XRPD peak positions in Table 9. In yet another embodiment, quinine sulfate polymorph Form J exhibits an X-ray powder diffraction pattern which is substantially similar to FIG. 9.

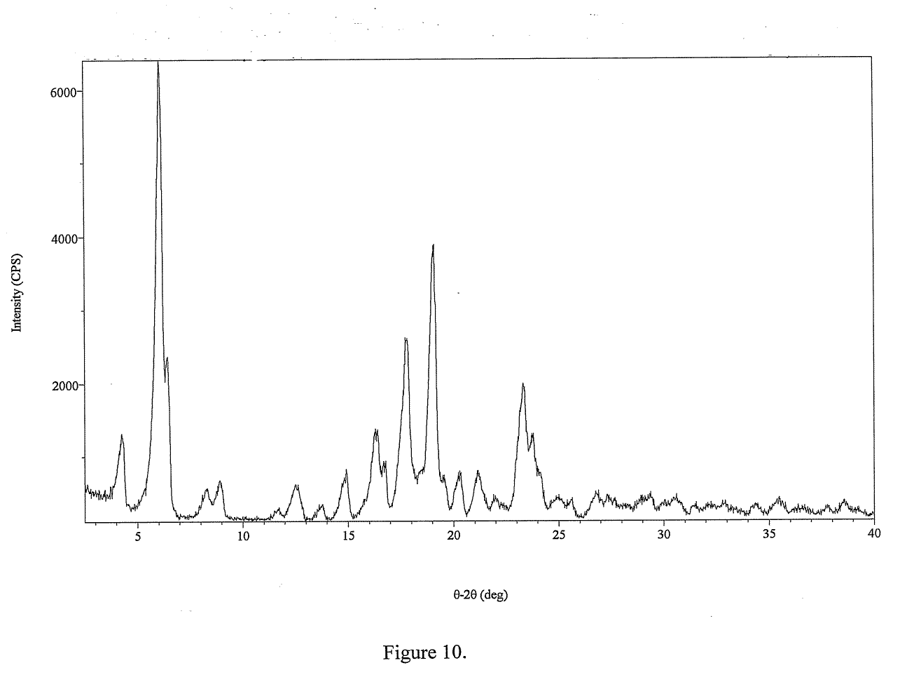

In one embodiment, quinine sulfate polymorph Form K exhibits XRPD peak positions at 4.3, 6.1, 6.4, 8.9, 17.7, 19.1, and 23.3 ±0.2 degrees 2-theta. In another embodiment, quinine sulfate polymorph Form K exhibits the XRPD peak positions in Table 10. In yet another embodiment, quinine sulfate polymorph Form K exhibits an X-ray powder diffraction pattern which is substantially similar to FIG. 10.

In another embodiment, quinine sulfate comprises non-crystalline quinine sulfate.

Processes of preparing quinine sulfate Forms B-K include crystallization or precipitation from a solvent system containing a single solvent or two or more solvents. Optionally, an anti-solvent can be used.

In a generalized procedure, quinine sulfate is dissolved in a solvent system with optional heating to form a crystallization solution. The heated solution can be at about the boiling point of the solvent system, specifically about 25 to about 100° C., more specifically about 30 to about 90° C., yet more specifically about 40 to about 80° C., and still yet more specifically about 50 to about 70° C.

The crystallization solution can be allowed to stand at ambient temperature or cooled to a lower temperature to allow crystal formation. Temperatures for crystal formation can be about −20 to about 25° C., specifically about −10 to about 20° C., more specifically about 0 to about 15° C., and yet more specifically about 3 to about 10° C.

The crystallization can be accomplished with slow cooling or rapid cooling. Rapid cooling can involve placing the crystallization solution under conditions of the targeted lower temperature without a gradual lowering of the temperature. Slow cooling can involve reducing the temperature of the crystallization solution at about 1 to about 30° C. per hour, specifically about 5 to about 25° C. per hour, and yet more specifically about 10 to about 20° C. per hour to a targeted lower temperature.

Optionally, the crystallization solution, prior to any solids formation, can be filtered to remove any undissolved solids, solid impurities and the like prior to removal of the solvent. Any filtration system and filtration techniques known in the art can be used.

In one embodiment, the crystallization solutions can be seeded with the desired polymorph Form.

Suitable solvents for preparing the crystalline forms of quinine sulfate include those that do not adversely affect the stability of the quinine sulfate, and are preferably inert. Suitable solvents may be organic, aqueous, or a mixture thereof. Suitable organic solvents may be aliphatic alcohols such as methanol (MeOH), ethanol (EtOH), n-propanol, isopropanol (IPA), n-butanol, tert-butanol (t-BuOH), tert-amyl alcohol (t-AmOH), 2,2,2-trifluoroethanol (TFE), and hexafluoroisopropanol (HFIPA); ethers such as tetrahydrofuran (THF), dioxane, methyl-tert-butyl ether, 1,2-dimethoxyethane (DME), and 2-methyl tetrahydrofuran; aliphatic ketones such as acetone, methyl ethyl ketone (MEK), and methyl isobutyl ketone; aliphatic carboxylic esters such as methyl acetate, ethyl acetate (EtOAc), and isopropyl acetate; aromatic hydrocarbons such as benzene, toluene, and xylene; aliphatic hydrocarbons such as hexane; aliphatic nitriles such as acetonitrile (MeCN); chlorinated hydrocarbons such as dichloromethane (DCM), chloroform, and carbon tetrachloride; aliphatic sulfoxides such as dimethyl sulfoxide (DMSO); amides such as dimethylformamide (DMF) and dimethylacetamide (DMA); organic acids such as acetic acid; N-methyl-2-pyrrolidone; pyridine; and the like, as well as mixtures comprising at least one of the foregoing organic solvents. Other solvents can be used as an anti-solvent to induce crystal formation of the quinine sulfate from solution. Exemplary anti-solvents include those solvents for which quinine sulfate is not readily soluble in, such as an aliphatic hydrocarbon including heptane, and the like; aqueous solvents including water and water combined with a water-miscible solvent, and the like; and combinations thereof.

“Solvent system” means a single or a combination of two or more solvents.

In one embodiment, quinine sulfate Form B is prepared by dissolving quinine sulfate in ethanol at about 15 to about 30° C. to form a solution and allowing the ethanol to evaporate until quinine Form B forms. The temperature can specifically be about 20 to about 25° C. and more specifically about 22 to about 24° C.

In another embodiment, quinine sulfate Form B is prepared by dissolving quinine sulfate in ethanol at about 31 to about 78° C., specifically about 40 to about 65° C., and more specifically about 50 to about 60° C. to form a solution, cooling the solution to about room temperature and allowing the quinine sulfate Form B to precipitate.

In one embodiment, quinine sulfate Form C is prepared by dissolving quinine sulfate in ethanol to form a solution and adding an aliphatic hydrocarbon antisolvent until quinine Form C forms. The antisolvent can be heptane.

In another embodiment, amorphous quinine is exposed to about 55 to about 65, specifically about 58 to about 62% relative humidity for about 19 days or more to form quinine sulfate Form C.

In one embodiment, quinine sulfate Form D is prepared by vacuum drying Form A at about 55 to about 65° C., specifically about 58 to about 62° C. for about 11 days or more, specifically about 11 to about 20 days to form quinine sulfate Form D.

In one embodiment, quinine sulfate Form E is prepared by dissolving quinine sulfate in methanol to form a solution and adding an aqueous antisolvent until quinine sulfate Form E forms. The antisolvent can be water.

In another embodiment, quinine sulfate Form E is prepared by dissolving quinine sulfate in 2,2,2-trifluoroethanol and water with heating to form a solution. The heated solution is allowed to cool gradually to about −10 to about 15° C., specifically about 0 to about 10° C., and yet more specifically about 2 to about 5° C. The ratio of 2,2,2-trifluoroethanol to water can be about 7:3 to about 3:7, specifically about 6:4 to about 4:6, and yet more specifically about 1:1.

In one embodiment, quinine sulfate Form F is prepared by slurrying quinine sulfate in ethanol for 5 days or greater, specifically about 5 to about 7 days; wherein the slurrying is carried out at a temperature of about 40 to about 60° C., specifically about 45 to about 55° C., and yet more specifically about 48 to about 52° C. Optionally, the ethanol is allowed to evaporate during the slurrying process.

In one embodiment, quinine sulfate Form G is prepared by slurrying quinine sulfate in tert-butanol for 3 days or greater, specifically about 3 to about 5 days; wherein the slurrying is carried out at a temperature of about 40 to about 60° C., specifically about 45 to about 55° C., and yet more specifically about 48 to about 52° C.

In one embodiment, quinine sulfate Form H is prepared by dissolving quinine sulfate in hexafluoroisopropanol and allowing the solvent to evaporate until quinine sulfate Form H forms.

In one embodiment, quinine sulfate Form I is prepared by slurrying quinine sulfate in methanol for 5 days or greater, specifically about 5 to about 7 days; wherein the slurrying is carried out at a temperature of about 40 to about 60° C., specifically about 45 to about 55° C., and yet more specifically about 48 to about 52° C. Optionally, the methanol is allowed to evaporate during the slurrying process.

In one embodiment, quinine sulfate Form J is prepared by suspending quinine sulfate in a combination of 1,1,1-trifluoroethanol and tetrahydrofuran to form a mixture, sonicating the mixture, and allowing the solvent to evaporate under ambient conditions. In another embodiment, the solvent is removed under reduced pressure. The ratio of 1,1,1-trifluoroethanol to tetrahydrofuran can be about 7:3 to about 3:7, specifically about 6:4 to about 4:6, and yet more specifically about 1:1.

In one embodiment, quinine sulfate Form K is prepared by slurrying quinine sulfate in tetrahydrofuran for 8 days or greater, specifically about 8 to about 10 days; wherein the slurrying is carried out at a temperature of about 40 to about 60° C., specifically about 45 to about 55° C., and yet more specifically about 48 to about 52° C.

In one embodiment, quinine sulfate non-crystalline form is prepared by preparing a saturated solution of quinine sulfate Form A in 1,1,1-trifluoroethanol at about 50 to about 70° C., specifically about 55 to about 65° C., and yet more specifically about 58 to about 62° C., cooling to about room temperature and removing the solvent by centrifugal evaporation under reduced pressure to form quinine sulfate non-crystalline form.

The polymorphs of quinine sulfate disclosed herein are useful to treat, for example, malaria caused by Plasmodium species, uncomplicated Plasmodium falciparum malaria, severe or complicated Plasmodium falciparum malaria, malaria caused by Plasmodium vivax, or for the prophylaxis of malaria caused by the foregoing; the treatment and prophylaxis of nocturnal recumbency leg muscle cramps, and the treatment of babesiosis caused by Babesia microti.

Malaria is a parasitic disease caused by the Plasmodium species P. falciparum, P. vivax, P. ovale and P. malariae. Malaria parasites are transmitted by female Anopheles mosquitoes. Without being held to theory, it is believed that quinine toxic to the malaria parasite, specifically by interfering with the parasite's ability to break down and digest hemoglobin, thus starving the parasite or causing the build-up of toxic levels of partially degraded hemoglobin.

Nocturnal recumbency leg muscle cramps, a common complaint in the elderly, is manifested as painful, involuntary contractions in the lower extremities after recumbency. Quinine can be used in the treatment of leg cramps including those associated with arthritis, diabetes, varicose veins, thrombophlebitis, arteriosclerosis and static foot deformities.

Babesiosis is caused by Babesia microti. The definitive host is a tick, in this case the deer tick, Ixodes dammini (I. scapularis). Humans enter the cycle when bitten by infected ticks. During a blood meal, a Babesia-infected tick introduces sporozoites into the human host.

Also disclosed herein are pharmaceutical compositions comprising the quinine sulfate polymorphic forms and non-crystalline form prepared herein. Excipients may be added to quinine sulfate compositions to facilitate manufacture, enhance stability, control release, enhance product characteristics, enhance bioavailability, enhance patient acceptability, etc. Pharmaceutically acceptable excipients include binders, disintegrants, lubricants, glidants, fillers, compression aids, colors, sweeteners, preservatives, suspending agents, dispersing agents, film formers, flavors, printing inks, etc. Binders hold the ingredients in the dosage form together. Exemplary binders include, for example, polyvinyl pyrrolidone, hydroxypropyl cellulose, hydroxypropyl methylcellulose, methylcellulose and hydroxyethyl cellulose, sugars, and combinations comprising one or more of the foregoing binders. Disintegrants expand when wet causing a tablet to break apart. Exemplary disintegrants include water swellable substances, for example, low-substituted hydroxypropyl cellulose; cross-linked polyvinyl pyrrolidone (PVP-XL); cross-linked sodium carboxymethylcellulose (sodium croscarmellose); sodium starch glycolate; sodium carboxymethylcellulose; sodium carboxymethyl starch; ion-exchange resins; microcrystalline cellulose; starches and pregelatinized starch; formalin-casein, and combinations comprising one or more of the foregoing water swellable substances. Lubricants, for example, aid in the processing of powder materials. Exemplary lubricants include calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants. Glidants include, for example, silicon dioxide. The filler may be a water insoluble filler, such as silicon dioxide, titanium dioxide, talc, alumina, starch, kaolin, polacrilin potassium, powdered cellulose, microcrystalline cellulose, and combinations comprising one or more of the foregoing fillers. Exemplary water-soluble fillers include water soluble sugars and sugar alcohols, preferably lactose, glucose, fructose, sucrose, mannose, dextrose, galactose, the corresponding sugar alcohols and other sugar alcohols, such as mannitol, sorbitol, xylitol, and combinations comprising one or more of the foregoing fillers.

Solid dosage forms for oral administration include, but are not limited to, capsules, tablets, powders, and granules. In such solid dosage forms, the active agent may be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, and combinations comprising one or more of the foregoing additives. For capsules, tablets, and pills, the dosage forms may also comprise buffering agents.

The following examples further illustrate the invention but, of course, should not be construed as in any way limiting its scope.

### EXAMPLES

### Example 1

### Preparation of Quinine Sulfate Form B

Quinine sulfate (30.1 mg) solids are added to a vial. Ethanol (2 mL) is added and solids dissolved. The sample is filtered through a 0.2 μm nylon filter and left uncapped under ambient conditions for evaporation. Quinine sulfate Form B spontaneously precipitates from solution at high temperature. The resulting solids exhibit a unique XRPD powder pattern designated Form B (Table 1, FIG. 1). Infrared and Raman peak positions are provided in Table 1.

XRPD analyses are performed using an Inel XRG-3000 XRG-3000 diffractometer equipped with a CPS (Curved Position Sensitive) detector with a 2θ range of 120°. Real time data are collected using Cu—Kα radiation. The tube voltage and amperage are set to 40 kV and 30 mA, respectively. The monochromator slit is set at 5 mm by 160 μm. The patterns are displayed from 2.5-40 °2θ. Samples are prepared for analysis by packing them into thin-walled glass capillaries. Each capillary is mounted onto a goniometer head that is motorized to permit spinning of the capillary during data acquisition. The samples are analyzed for either 300 sec or 600 sec. Instrument calibration is performed using a silicon reference standard.

XRPD analyses are also performed using a Shimadzu XRD-6000 X-ray powder diffractometer using Cu Kα radiation. The instrument is equipped with a long fine focus X-ray tube. The tube voltage and amperage are set to 40 kV and 40 mA, respectively. The divergence and scattering slits are set at 1° and the receiving slit is set at 0.15 mm. Diffracted radiation is detected by a NaI scintillation detector. A θ-2θ continuous scan at 3°/min (0.4 sec/0.02° step) from 2.5 to 40° 2θ is used. A silicon standard is analyzed to check the instrument alignment. Data are collected and analyzed using XRD-6100/7000 v. 5.0. Samples are prepared for analysis by placing them in an aluminum holder with silicon insert.

Infrared spectra are acquired on a Magna-IR 860® Fourier Transform Infrared (FTIR) spectrophotometer (Thermo Nicolet) equipped with an Ever-Glo mid/far IR source, an extended range potassium bromide (KBr) beamsplitter, and a deuterated triglycine sulfate (DTGS) detector. An ATR Thunderdome accessory with a standard tip is used for sampling. Sample preparation consists of placing the sample on a germanium crystal and pressing the material against the crystal using a plunger. Each spectrum represents 256 co-added scans collected at a spectral resolution of 4 cm⁻¹. A Log 1/R (R=reflectance) spectrum is acquired by taking a ratio of these two data sets against each other. Wavelength calibration is performed using styrene.

FT-Raman spectra are acquired on a FT-Raman 960 spectrometer (Thermo Nicolet). This spectrometer uses an excitation wavelength of 1064 nm. Approximately 0.5-0.8 W of Nd:YVO₄ laser power is used to irradiate the sample. The Raman spectra are measured with an indium gallium arsenide (InGaAs) detector. The samples are prepared for analysis by placing the material in a glass capillary and positioning the capillary in a gold-coated tube holder in the accessory. A total of 256 sample scans are collected from 3600-100 cm⁻¹ at a spectral resolution of 4 cm⁻¹, using Happ-Genzel apodization. Wavelength calibration is performed using sulfur and cyclohexane.

TABLE 1
XRPD Peak Positions, Infrared Peak Positions and Raman Peak Positions
for Quinine Sulfate Form B

| XRPD Peak Position | Rel. Intensity of | Infrared Peak | Raman peak |
|---|---|---|---|
| (°2θ) | XRPD peak (%) | (cm⁻¹) | (cm⁻¹) |
| 6.1 | 6 | 1620 | 3076 |
| 8.3 | 35 | 1593 | 3046 |
| 8.6 | 7 | 1509 | 2992 |
| 10.3 | 1 | 1473 | 2976 |
| 11.9 | 22 | 1433 | 2955 |
| 12.2 | 22 | 1365 | 2837 |
| 14.5 | 6 | 1345 | 1644 |
| 14.7 | 9 | 1322 | 1618 |
| 15.4 | 8 | 1303 | 1592 |
| 16.8 | 46 | 1243 | 1574 |
| 17.3 | 100 | 1141 | 1508 |
| 17.7 | 12 | 1082 | 1457 |
| 18.5 | 13 | 1028 | 1444 |
| 19.3 | 5 | 1003 | 1430 |
| 20.6 | 38 | 981 | 1363 |
| 21.8 | 8 | 941 | 1312 |
| 22.6 | 6 | 918 | 1228 |
| 22.9 | 5 | 881 | 1202 |
| 23.9 | 11 | 858 | 1178 |
| 24.5 | 27 | 829 | 1132 |
| 24.8 | 15 | 803 | 1078 |
| 25.7 | 18 | 761 | 1055 |
| 26.1 | 16 | 719 | 976 |
| 26.6 | 9 | | 924 |
| 27.2 | 2 | | 855 |
| 27.9 | 3 | | 832 |
| 28.8 | 5 | | 802 |
| 29.3 | 9 | | 789 |
| 31.1 | 2 | | 775 |
| 32.3 | 4 | | 763 |
| 33.0 | 8 | | |
| 34.1 | 3 | | |
| 35.1 | 4 | | |
| 36.2 | 2 | | |
| 37.1 | 6 | | |
| 39.1 | 4 | | |
| 39.9 | 5 | | |

Quinine sulfate Form B is analyzed by X-ray powder diffraction (XRPD), differential scanning calorimetry (DSC), and thermogravimetric analysis (TGA). Quinine sulfate Form B exhibits endotherms at 123° C. and 225° C. and an exotherm at 229° C. The material exhibits a 1.5% weight loss from 94-141° C.

Differential scanning calorimetry (DSC) is performed using a TA Instruments model 2920 calorimeter. The sample is placed into an aluminum DSC pan, the weight accurately recorded, and the pan is crimped. The sample cell is equilibrated at 25° C. and heated under a nitrogen purge at a rate of 10° C./min, up to a final temperature of 250° C. Indium metal is used as the calibration standard.

Thermogravimetric analyses (TGA) are performed using a TA Instruments 2950 thermogravimetric analyzer. Each sample is placed in an aluminum sample pan and inserted into the TG furnace. The furnace is first equilibrated at 25° C., then heated under nitrogen at a rate of 10° C./min, up to a final temperature of 350° C. Nickel and Alumel™ are used as the calibration standards.

### Example 2

### Preparation of Quinine Sulfate Form C

A saturated solution of quinine sulfate is prepared in ethanol (1 mL). The mixture is filtered through a 0.2 μm nylon filter into a vial containing heptane (10 mL) and precipitation occurs (“crash precipitation”). The resulting mixture is filtered through filter paper and solids collected in vial. The resulting solids exhibit a unique XRPD powder pattern designated Form C (Table 2, FIG. 2). Infrared and Raman peak positions are provided in Table 2.

TABLE 2
XRPD Peak Positions, Infrared Peak Positions and Raman Peak Positions
for Quinine Sulfate Form C

| XRPD Peak Position | Rel. Intensity of | Infrared Peak | Raman peak |
|---|---|---|---|
| (°2θ) | XRPD peak (%) | (cm⁻¹) | (cm⁻¹) |
| 4.2 | 4 | 1618 | 3072 |
| 5.2 | 4 | 1592 | 3047 |
| 6.2 | 84 | 1508 | 2950 |
| 8.2 | 4 | 1472 | 2909 |
| 9.2 | 85 | 1432 | 2882 |
| 12.4 | 15 | 1364 | 2846 |
| 12.9 | 50 | 1344 | 1641 |
| 14.0 | 48 | 1318 | 1618 |
| 15.3 | 34 | 1303 | 1592 |
| 16.6 | 39 | 1261 | 1574 |
| 17.5 | 100 | 1240 | 1504 |
| 18.4 | 49 | 1182 | 1461 |
| 18.7 | 76 | 1128 | 1444 |
| 19.1 | 73 | 1081 | 1432 |
| 19.5 | 49 | 1022 | 1366 |
| 20.7 | 38 | 1004 | 1304 |
| 20.9 | 45 | 982 | 1227 |
| 21.6 | 14 | 931 | 1203 |
| 22.0 | 40 | 858 | 1185 |
| 22.4 | 41 | 831 | 1125 |
| 23.3 | 50 | 804 | 1076 |
| 24.5 | 11 | 763 | 1056 |
| 24.9 | 9 | 719 | 967 |
| 25.9 | 34 | | 919 |
| 26.7 | 43 | | 853 |
| 27.3 | 33 | | 834 |
| 28.2 | 14 | | 803 |
| 28.9 | 16 | | 790 |
| 29.7 | 6 | | 775 |
| 30.9 | 15 | | 765 |
| 31.3 | 11 | | |
| 32.4 | 5 | | |
| 33.5 | 5 | | |
| 34.1 | 10 | | |
| 35.6 | 7 | | |
| 36.0 | 8 | | |
| 37.5 | 6 | | |
| 38.7 | 5 | | |

Quinine sulfate Form C is analyzed by X-ray powder diffraction (XRPD) and Thermogravimetric/infrared analysis (TG-IR). TG-IR analysis shows continuous weight loss from 25 to 225° C. indicating possible water loss.

### Example 3

### Preparation of Quinine Sulfate Form D

A sample of quinine sulfate Form A is added to a capillary tube and placed in a vacuum oven set at 60° C. After 11 days the solid material is removed and analyzed by XRPD. The resulting solids exhibited a unique XRPD powder pattern designated Form D (Table 3, FIG. 3).

TABLE 3
XRPD Peak Positions for Quinine Sulfate Form D

| XRPD Peak Position | Rel. Intensity of XRPD |
|---|---|
| (°2θ) | peak (%) |
| 6.4 | 4 |
| 8.6 | 100 |
| 9.7 | 18 |
| 11.7 | 6 |
| 13.2 | 2 |
| 14.1 | 48 |
| 14.7 | 5 |
| 16.2 | 18 |
| 16.8 | 81 |
| 17.2 | 19 |
| 17.6 | 14 |
| 18.1 | 23 |
| 18.6 | 13 |
| 18.9 | 20 |
| 19.5 | 14 |
| 19.9 | 54 |
| 20.5 | 6 |
| 21.3 | 14 |
| 22.1 | 5 |
| 22.7 | 22 |
| 23.1 | 5 |
| 23.4 | 6 |
| 24.2 | 4 |
| 24.8 | 8 |
| 25.2 | 13 |
| 25.8 | 20 |
| 26.8 | 11 |
| 28.1 | 7 |
| 29.7 | 3 |
| 30.2 | 3 |
| 31.2 | 3 |
| 31.7 | 2 |
| 33.3 | 4 |
| 33.7 | 5 |
| 34.9 | 2 |
| 35.5 | 2 |
| 36.2 | 2 |
| 37.0 | 2 |
| 37.9 | 3 |
| 38.7 | 2 |

### Example 4

### Preparation of Quinine Sulfate Form E

A saturated solution of quinine sulfate is prepared in methanol (2 mL) and warmed on a hotplate set at 75° C. The mixture is filtered through a 0.2 μm nylon filter into a vial containing water (10 mL) and precipitation occurs (“crash precipitation”). The resulting mixture is filtered through filter paper and solids collected in vial. The resulting solids exhibit a unique XRPD powder pattern designated Form E (Table 4, FIG. 4). Infrared and Raman peak positions are provided in Table 4.

TABLE 4
XRPD Peak Positions, Infrared Peak Positions and Raman Peak Positions
for Quinine Sulfate Form E

| XRPD Peak Position | Rel. Intensity of | Infrared Peak | Raman peak |
|---|---|---|---|
| (°2θ) | XRPD peak (%) | (cm⁻¹) | (cm⁻¹) |
| 5.5 | 5 | 1620 | 3077 |
| 8.3 | 83 | 1594 | 3047 |
| 9.0 | 5 | 1510 | 2999 |
| 9.5 | 7 | 1474 | 2973 |
| 10.4 | 10 | 1433 | 2904 |
| 11.1 | 7 | 1366 | 2843 |
| 13.2 | 4 | 1345 | 1647 |
| 14.4 | 100 | 1323 | 1620 |
| 16.2 | 79 | 1303 | 1595 |
| 16.6 | 21 | 1243 | 1575 |
| 17.0 | 18 | 1141 | 1510 |
| 17.3 | 10 | 1082 | 1460 |
| 17.9 | 24 | 1027 | 1433 |
| 18.4 | 29 | 1002 | 1365 |
| 18.8 | 60 | 981 | 1313 |
| 19.3 | 21 | 952 | 1230 |
| 19.7 | 26 | 941 | 1205 |
| 20.1 | 23 | 918 | 1179 |
| 20.8 | 16 | 883 | 1132 |
| 22.4 | 65 | 858 | 1083 |
| 23.0 | 4 | 828 | 1055 |
| 23.8 | 7 | 803 | 965 |
| 24.4 | 12 | 761 | 926 |
| 24.8 | 44 | 719 | 857 |
| 25.2 | 24 | | 835 |
| 25.6 | 26 | | 803 |
| 26.0 | 36 | | 773 |
| 26.8 | 6 | | 763 |
| 27.7 | 30 | | |
| 28.5 | 22 | | |
| 29.2 | 25 | | |
| 29.5 | 20 | | |
| 30.5 | 16 | | |
| 31.0 | 16 | | |
| 32.1 | 24 | | |
| 32.7 | 13 | | |
| 33.6 | 6 | | |
| 34.2 | 6 | | |
| 36.5 | 7 | | |
| 37.0 | 5 | | |
| 39.7 | 5 | | |

Quinine sulfate Form E is analyzed by X-ray powder diffraction (XRPD) and Thermogravimetric/infrared analysis (TG-IR). TG-IR analysis shows 25% weight loss from 18 to 55° C. and a 4% weight loss from 55-85° C. indicating possible water loss.

### Example 5

### Preparation of Quinine Sulfate Form F

A saturated solution of quinine sulfate Form A material is prepared in ethanol (3 mL). The sample is briefly placed onto a hotplate set at 75° C. Additional ethanol (7 mL) is added and solids persisted. The sample is allowed to cool to room temperature before being placed into an orbit shaker set at 50° C. The resulting mixture is agitated (slurried) for 5 days. Evaporation of the solvent occurred causing dried solids to form at the top of the vial. The remaining solvent is decanted and the solids blotted dry with filter paper. The resulting solids exhibit a unique XRPD powder pattern designated Form F (Table 5, FIG. 5). Infrared and Raman peak positions are provided in Table 5.

TABLE 5
XRPD Peak Positions, Infrared Peak Positions and Raman Peak Positions
for Quinine Sulfate Form F

| XRPD Peak Position | Rel. Intensity of | Infrared Peak | Raman peak |
|---|---|---|---|
| (°2θ) | XRPD peak (%) | (cm⁻¹) | (cm⁻¹) |
| 6.9 | 4 | 1620 | 3078 |
| 7.5 | 100 | 1594 | 3045 |
| 8.3 | 70 | 1509 | 2999 |
| 8.5 | 17 | 1473 | 2974 |
| 11.8 | 4 | 1433 | 2963 |
| 13.1 | 8 | 1366 | 2841 |
| 14.5 | 4 | 1345 | 1645 |
| 15.0 | 16 | 1322 | 1619 |
| 15.4 | 28 | 1303 | 1593 |
| 17.0 | 24 | 1243 | 1575 |
| 17.5 | 41 | 1141 | 1509 |
| 18.1 | 7 | 1082 | 1460 |
| 18.6 | 8 | 1028 | 1445 |
| 19.0 | 6 | 1003 | 1432 |
| 19.9 | 8 | 981 | 1364 |
| 20.6 | 18 | 941 | 1313 |
| 21.4 | 4 | 918 | 1229 |
| 22.1 | 6 | 882 | 1204 |
| 22.5 | 11 | 858 | 1179 |
| 22.8 | 7 | 828 | 1132 |
| 23.2 | 4 | 804 | 1081 |
| 24.8 | 14 | 760 | 1056 |
| 25.1 | 12 | 719 | 977 |
| 25.4 | 8 | | 966 |
| 25.8 | 7 | | 925 |
| 26.5 | 7 | | 956 |
| 28.3 | 4 | | 832 |
| 29.7 | 3 | | 803 |
| 30.5 | 3 | | 789 |
| 31.0 | 3 | | 774 |
| 31.5 | 3 | | 762 |
| 33.2 | 3 | | |
| 34.2 | 5 | | |
| 36.3 | 2 | | |
| 36.8 | 3 | | |
| 38.5 | 2 | | |

Quinine sulfate Form F is analyzed by X-ray powder diffraction (XRPD) and Thermogravimetric/infrared analysis (TG-IR). TG-IR analysis shows 5% weight loss from 16-80° C. and 1% weight loss from 80-135° C.

### Example 6

### Preparation of Quinine Sulfate Form G

A saturated solution of quinine sulfate is prepared in t-butanol (6 mL). This mixture is agitated (slurried) for 3 days in an orbital shaker at 50° C. After 3 days, the solvent is decanted off and the solids blotted dry with filter paper. The resulting solids exhibit a unique XRPD powder pattern designated Form G (Table 6, FIG. 6). Infrared and Raman peak positions are provided in Table 6.

TABLE 6
XRPD Peak Positions, Infrared Peak Positions and Raman Peak Positions
for Quinine Sulfate Form G

| XRPD | Rel. Intensity of | Infrared Peak | Raman peak |
|---|---|---|---|
| Peak Position (°2θ) | XRPD peak (%) | (cm⁻¹) | (cm⁻¹) |
| 3.2 | 4 | 1619 | 3077 |
| 4.2 | 2 | 1591 | 3047 |
| 6.1 | 100 | 1508 | 2993 |
| 7.3 | 8 | 1473 | 2976 |
| 7.7 | 14 | 1431 | 2958 |
| 9.9 | 1 | 1362 | 2885 |
| 10.3 | 2 | 1343 | 2839 |
| 12.0 | 2 | 1319 | 1644 |
| 12.7 | 5 | 1304 | 1618 |
| 13.1 | 4 | 1242 | 1592 |
| 15.1 | 2 | 1132 | 1574 |
| 15.4 | 3 | 1064 | 1509 |
| 16.8 | 14 | 1028 | 1445 |
| 17.9 | 31 | 1007 | 1431 |
| 18.8 | 21 | 981 | 1363 |
| 20.2 | 7 | 950 | 1313 |
| 20.7 | 3 | 930 | 1228 |
| 21.0 | 5 | 880 | 1203 |
| 22.5 | 4 | 857 | 1177 |
| 22.9 | 4 | 832 | 1132 |
| 23.4 | 5 | 802 | 1080 |
| 23.9 | 4 | 762 | 1056 |
| 24.7 | 3 | 719 | 977 |
| 25.1 | 2 | | 924 |
| 26.3 | 1 | | 856 |
| 27.2 | 4 | | 831 |
| 28.3 | 2 | | 789 |
| 29.1 | 2 | | 774 |
| 29.7 | 2 | | 762 |
| 31.4 | 1 | | |
| 32.4 | 3 | | |
| 35.3 | 5 | | |
| 36.3 | 5 | | |

Quinine sulfate Form G is analyzed by X-ray powder diffraction (XRPD) and Thermogravimetric/infrared analysis (TG-IR). TG-IR analysis shows 8% weight loss from 19-150° C. indicating possible bound t-butanol.

### Example 7

### Preparation of Quinine Sulfate Form H

Quinine sulfate (24.6 mg) solids are added to a vial. Hexafluoroisopropanol (0.5 mL) is added and the solids dissolved. The sample is filtered through 0.2 μm nylon filter and left uncapped under ambient conditions for evaporation. The resulting solids exhibit a unique XRPD powder pattern designated Form H (Table 7, FIG. 7). Infrared and Raman peak positions are provided in Table 7.

TABLE 7
XRPD Peak Positions, Infrared Peak Positions and Raman Peak Positions
for Quinine Sulfate Form H

| XRPD | Rel. Intensity of | Infrared Peak | Raman peak |
|---|---|---|---|
| Peak Position (°2θ) | XRPD peak (%) | (cm⁻¹) | (cm⁻¹) |
| 4.5 | 7 | 1619 | 3076 |
| 5.9 | 43 | 1591 | 3046 |
| 6.4 | 38 | 1508 | 2974 |
| 7.9 | 63 | 1473 | 2956 |
| 9.1 | 18 | 1430 | 1643 |
| 10.5 | 10 | 1363 | 1619 |
| 11.7 | 18 | 1342 | 1591 |
| 13.9 | 23 | 1318 | 1574 |
| 14.5 | 23 | 1289 | 1509 |
| 14.9 | 23 | 1256 | 1445 |
| 15.8 | 46 | 1241 | 1430 |
| 16.5 | 57 | 1177 | 1363 |
| 16.8 | 54 | 1150 | 1312 |
| 17.2 | 26 | 1133 | 1228 |
| 17.8 | 61 | 1080 | 1204 |
| 18.1 | 100 | 1054 | 1177 |
| 19.4 | 63 | 1028 | 1133 |
| 19.7 | 61 | 984 | 1077 |
| 21.0 | 22 | 957 | 1055 |
| 21.5 | 12 | 930 | 970 |
| 22.3 | 13 | 857 | 923 |
| 23.2 | 37 | 832 | 856 |
| 24.0 | 19 | 802 | 832 |
| 24.8 | 8 | 763 | 802 |
| 25.2 | 6 | 736 | 789 |
| 25.8 | 5 | 719 | 775 |
| 27.6 | 8 | | 764 |
| 29.3 | 6 | | |
| 30.2 | 8 | | |
| 31.5 | 6 | | |
| 33.6 | 4 | | |
| 34.5 | 3 | | |
| 37.1 | 3 | | |
| 38.1 | 3 | | |

Quinine sulfate Form H is analyzed by X-ray powder diffraction (XRPD) and Thermogravimetric/infrared analysis (TG-IR). TG-IR analysis shows 11% weight loss from 19-150° C. indicating possible hexafluoroisopropanol solvate.

### Example 8

### Preparation of Quinine Sulfate Form I

A saturated solution of quinine sulfate Form A material is prepared in methanol (1 mL). This mixture is agitated (slurried) for 5 days in an orbital shaker at 50° C. Evaporation of the solvent occurred, leaving behind dried solids at the top of the vial. The solvent is decanted off and the solids blotted dry with filter paper. The resulting solids exhibit a unique XRPD powder pattern designated Form I (Table 8, FIG. 8). Infrared and Raman peak positions are provided in Table 8.

TABLE 8
XRPD XRPD Peak Positions, Infrared Peak Positions and Raman Peak
Positions for Quinine Sulfate Form I

| XRPD Peak Position | Rel. Intensity of | Infrared Peak | Raman peak |
|---|---|---|---|
| (°2θ) | XRPD peak (%) | (cm⁻¹) | (cm⁻¹) |
| 8.3 | 100 | 1620 | 3078 |
| 9.9 | 6 | 1594 | 3045 |
| 10.5 | 11 | 1510 | 2998 |
| 11.7 | 8 | 1474 | 2974 |
| 14.0 | 16 | 1433 | 2903 |
| 14.2 | 13 | 1366 | 2839 |
| 14.8 | 51 | 1345 | 1647 |
| 15.6 | 19 | 1323 | 1620 |
| 16.6 | 2 | 1303 | 1595 |
| 17.2 | 17 | 1243 | 1576 |
| 17.6 | 29 | 1141 | 1510 |
| 17.9 | 13 | 1082 | 1461 |
| 18.4 | 6 | 1028 | 1433 |
| 18.9 | 22 | 1003 | 1366 |
| 19.2 | 24 | 981 | 1314 |
| 19.9 | 7 | 952 | 1230 |
| 20.9 | 31 | 941 | 1206 |
| 21.6 | 11 | 918 | 1180 |
| 22.0 | 17 | 883 | 1132 |
| 23.5 | 7 | 858 | 1082 |
| 24.5 | 4 | 828 | 1056 |
| 25.1 | 17 | 804 | 966 |
| 25.4 | 6 | 760 | 926 |
| 26.1 | 14 | 719 | 858 |
| 26.6 | 11 | | 835 |
| 26.9 | 11 | | 804 |
| 27.5 | 6 | | 790 |
| 28.2 | 9 | | 773 |
| 28.6 | 12 | | 763 |
| 29.2 | 3 | | |
| 29.8 | 6 | | |
| 30.2 | 9 | | |
| 30.5 | 9 | | |
| 31.5 | 2 | | |
| 32.1 | 4 | | |
| 32.8 | 5 | | |
| 33.5 | 3 | | |
| 34.3 | 1 | | |
| 35.6 | 1 | | |
| 36.7 | 3 | | |
| 37.8 | 2 | | |
| 38.5 | 3 | | |

Quinine sulfate Form I is analyzed by X-ray powder diffraction (XRPD) and Thermogravimetric/infrared analysis (TG-IR). TG-IR analysis shows 4% weight loss from 41-81° C. indicating a hydrate (2 mol of water).

### Example 9

### Preparation of Quinine Sulfate Form J

Quinine sulfate solids (about 3 mg) are added to a microplate well. 1,1,1-Trifluoroethanol (about 0.03 mL) and tetrahydrofuran (about 0.03 mL) are added, the well is sonicated and solvent removed under reduced pressure. The resulting solids exhibit a unique XRPD powder pattern designated Form J (Table 9, FIG. 9).

Solids in well-plates are analyzed using a Bruker D-8 Discover diffractometer and Bruker's General Area Diffraction Detection System (GADDS, v. 4.1.20). An incident beam of Cu Kα radiation is produced using a fine-focus tube (40 kV, 40 mA), a Göbel mirror, and a 0.5 mm double-pinhole collimator. The samples are positioned for analysis by securing the well plate to a translation stage and moving each sample to intersect the incident beam. The samples are analyzed using a transmission geometry. The incident beam is scanned and rastered over the sample during the analysis to optimize orientation statistics. A beam-stop is used to minimize air scatter from the incident beam at low angles. Diffraction patterns are collected using a Hi-Star area detector located 15 cm from the sample and processed using GADDS. The intensity in the GADDS image of the diffraction pattern is integrated using a step size of 0.04° 2θ. The integrated patterns display diffraction intensity as a function of 2θ. Prior to the analysis a silicon standard is analyzed to verify the Si 111 peak position.

TABLE 9
XRPD Peak Positions for Quinine Sulfate Form J

| XRPD Peak Position | Rel. Intensity of XRPD peak |
|---|---|
| (°2θ) | (%) |
| 4.2 | 35 |
| 6.1 | 38 |
| 8.3 | 5 |
| 9.3 | 4 |
| 12.5 | 4 |
| 14.1 | 5 |
| 14.8 | 14 |
| 15.8 | 23 |
| 16.6 | 17 |
| 17.8 | 22 |
| 18.4 | 100 |
| 19.1 | 63 |
| 20.4 | 39 |
| 21.4 | 24 |
| 23.6 | 19 |
| 25.0 | 4 |
| 25.8 | 3 |
| 27.0 | 9 |
| 28.3 | 41 |
| 29.0 | 23 |
| 29.7 | 2 |
| 31.0 | 40 |
| 31.6 | 33 |
| 32.4 | 3 |
| 34.5 | 3 |
| 35.6 | 1 |

### Example 10

### Preparation of Quinine Sulfate Form K

A saturated solution of quinine sulfate Form A is prepared in tetrahydrofuran (2 mL). This mixture is agitated (slurried) for 8 days in an orbital shaker at 50° C. The resulting mixture is filtered through filter paper and solids collected in vial. The resulting solids exhibit a unique XRPD powder pattern designated Form K (Table 10, FIG. 10). Infrared and Raman peak positions are provided in Table 10.

TABLE 10
XRPD XRPD Peak Positions, Infrared Peak Positions and Raman Peak
Positions for Quinine Sulfate Form K

| XRPD Peak Position | Rel. Intensity of | Infrared Peak | Raman peak |
|---|---|---|---|
| (°2θ) | XRPD peak (%) | (cm⁻¹) | (cm⁻¹) |
| 4.3 | 11 | 1620 | 3075 |
| 6.1 | 100 | 1591 | 3051 |
| 6.4 | 36 | 1510 | 2997 |
| 8.3 | 7 | 1475 | 2974 |
| 8.9 | 9 | 1430 | 2957 |
| 11.7 | 3 | 1364 | 2889 |
| 12.5 | 9 | 1323 | 2872 |
| 13.8 | 4 | 1242 | 1641 |
| 14.9 | 11 | 1170 | 1620 |
| 16.3 | 20 | 1126 | 1591 |
| 16.8 | 13 | 1078 | 1574 |
| 17.7 | 40 | 1049 | 1510 |
| 19.1 | 60 | 1029 | 1446 |
| 19.5 | 10 | 1002 | 1434 |
| 20.3 | 9 | 986 | 1391 |
| 21.1 | 10 | 951 | 1363 |
| 22.0 | 3 | 932 | 1307 |
| 23.3 | 30 | 882 | 1230 |
| 23.8 | 18 | 858 | 1203 |
| 25.0 | 6 | 831 | 1178 |
| 25.6 | 5 | 803 | 1134 |
| 26.8 | 5 | 765 | 1080 |
| 27.3 | 6 | 719 | 1057 |
| 27.7 | 5 | | 971 |
| 29.3 | 4 | | 915 |
| 30.6 | 4 | | 858 |
| 32.9 | 3 | | 835 |
| 34.3 | 3 | | 803 |
| 35.5 | 4 | | 787 |
| 37.8 | 3 | | 775 |
| 38.6 | 3 | | 766 |

Quinine sulfate Form K is analyzed by X-ray powder diffraction (XRPD) and Thermogravimetric/infrared analysis (TG-IR). TG-IR analysis shows 3% weight loss from 18 to 118° C. and a 6% weight loss from 118-147° C. indicating possible evolution of tetrahydrofuran.

### Example 11

### Preparation of Quinine Sulfate Non-Crystalline

A saturated solution of quinine sulfate Form A is prepared in 1,1,1-trifluoroethanol (about 0.500 mL). This material is then warmed on a hot plate set at 60° C. and filtered through a 0.2 μm nylon filter. The solution is then seeded with quinine sulfate Form J and allowed to cool to room temperature. The solvent is then removed by centrifugal evaporation. The resulting solids exhibit a XRPD pattern characteristic of non-crystalline material.

### Quinine Sulfate Polymorph Form A

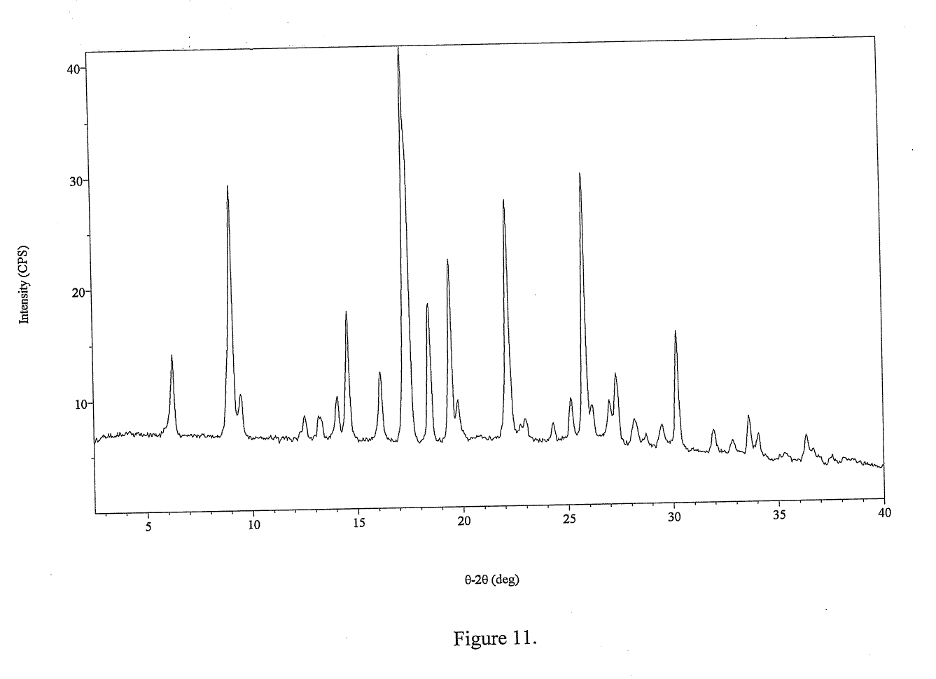

Quinine sulfate polymorph Form A exhibits the XRPD pattern as shown in Table 11a and FIG. 11. Infrared and Raman peak positions are provided in Table 11a.

TABLE 11a
XRPD Peak Positions, Infrared Peak Positions and Raman Peak Positions
for Quinine Sulfate Form A

| XRPD Peak Position | Rel. Intensity of | Infrared Peak | Raman peak |
|---|---|---|---|
| (°2θ) | XRPD peak (%) | (cm⁻¹) | (cm⁻¹) |
| 6.3 | 21 | 1620 | 3078 |
| 9.1 | 72 | 1594 | 3043 |
| 9.5 | 25 | 1509 | 2998 |
| 12.5 | 6 | 1474 | 2974 |
| 13.2 | 6 | 1433 | 2952 |
| 14.1 | 11 | 1366 | 2839 |
| 14.6 | 33 | 1345 | 1647 |
| 16.1 | 18 | 1323 | 1621 |
| 17.4 | 100 | 1303 | 1595 |
| 18.5 | 35 | 1258 | 1575 |
| 19.5 | 55 | 1243 | 1510 |
| 19.8 | 23 | 1212 | 1461 |
| 22.2 | 63 | 1141 | 1433 |
| 22.8 | 18 | 1082 | 1365 |
| 23.0 | 19 | 1028 | 1313 |
| 24.3 | 5 | 1002 | 1230 |
| 25.2 | 11 | 981 | 1205 |
| 25.8 | 72 | 952 | 1180 |
| 26.2 | 22 | 941 | 1132 |
| 27.0 | 23 | 918 | 1083 |
| 27.3 | 28 | 882 | 1055 |
| 28.2 | 7 | 858 | 965 |
| 28.7 | 15 | 828 | 857 |
| 29.5 | 6 | 804 | 804 |
| 30.2 | 30 | 761 | 773 |
| 32.0 | 6 | 719 | 763 |
| 32.8 | 4 | | |
| 33.6 | 19 | | |
| 34.1 | 15 | | |
| 35.3 | 10 | | |
| 36.3 | 14 | | |
| 36.7 | 11 | | |
| 37.6 | 2 | | |

In addition to XRPD, quinine sulfate polymorph A is analyzed by DSC, TGA, moisture balance (MB), post-MB XRPD, hotstage microscopy (HSM), Karl Fischer (KF) and variable temperature X-ray powder diffraction (VT-XRPD); a summary of the analyses is provided in Table 11b.

TABLE 11b

| Analysis | Result |
|---|---|
| XRPD | Form A |
| DSC | endotherms: 137° C. and 230° C. exotherm: 234° C. |
| TGA | 5% wt loss from 25-97° C., 2% weight loss |
| | from 217-251° C. |
| MB | non-hygroscopic |
| post-MB XRPD | Form A |
| HSM | melt onset: 221° C. |
| KF | water content: 4.61%, 4.59%, 4.66%, avg = 4.62% |
| VT-XRPD | ambient temp: Form A |
| | 50° C.: Form A |
| | 75° C.: Form A |
| | 110° C.: Pattern D |
| | 40° C.: Form A |

Moisture sorption/desorption data are collected on a VTI SGA-100 Vapor Sorption Analyzer. Sorption and desorption data are collected over a range of 5% to 95% relative humidity (RH) at 10% RH intervals. Samples are not dried prior to analysis. Equilibrium criteria used for analysis are less than 0.0100% weight change in 5 minutes, with a maximum equilibration time of 3 hours if the weight criterion is not met. Data are not corrected for the initial moisture content of the samples. Sodium chloride and polyvinylpyrrolidone are used as calibration standards.

Hotstage microscopy (HSM) is performed using a Linkam hotstage (model FTIR 600) mounted on a Leica DMLP microscope. Samples are observed using crossed polarized light. Samples are sandwiched between coverslips and visually observed as the stage is heated. Images are captured using a SPOT Insight™ color digital camera with SPOT Software v. 4.5.9. The hotstage is calibrated using USP melting point standards.

Coulometric Karl Fischer analysis for water determination is performed using a Mettler Toledo DL39 Karl Fischer titrator. Approximately 40 mg of sample is placed in the KF titration vessel containing approximately 3.5 mL of Hydranal—Coulomat AD and mixed for 132 seconds to ensure dissolution. The sample is then titrated by means of a generator electrode which produces iodine by electrochemical oxidation: 2I⁻=>I₂₊₂e⁻. Two replicates are obtained to ensure reproducibility.

Variable-temperature XRPD is performed on a Shimadzu XRD-6000 X-ray powder diffractometer equipped with an Anton Paar HTK 1200 high temperature stage. The sample is packed in a ceramic holder and analyzed from 2.5 to 40 °2θ at 3°/min (0.4 sec/0.02° step). A silicon standard is analyzed to check the instrument alignment. Temperature calibration is performed using vanillin and sulfapyridine standards. Data are collected and analyzed using XRD-6000 v. 4.1.

The terms “comprising”, “having”, “including”, and “containing” are to be construed as open-ended terms (i.e., meaning “including, but not limited to”). The terms “a” and “an” do not denote a limitation of quantity, but rather denote the presence of at least one of the referenced item. The term “or” means “and/or”. The endpoints of all ranges directed to the same component or property are inclusive and independently combinable.

Unless defined otherwise, technical and scientific terms used herein have the same meaning as is commonly understood by one of skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A solid state form of quinine sulfate comprising:
Form B quinine sulfate;
Form C quinine sulfate;
Form D quinine sulfate;
Form E quinine sulfate;
Form F quinine sulfate;
Form G quinine sulfate;
Form H quinine sulfate;
Form I quinine sulfate;
Form J quinine sulfate;
Form K quinine sulfate;
or a noncrystalline form of quinine sulfate.

2. Form B quinine sulfate of claim 1, comprising:
XRPD peak positions at 8.3, 11.9, 12.2, 16.8, 17.3, 18.5, 20.6, 24.5, 25.7, and 26.1 ±0.2 degrees 2-theta;
peaks according to Table 1 ±0.2 degrees 2-theta; or
an X-ray powder diffraction pattern which is substantially similar to FIG. 1.

3. A process for preparing quinine sulfate Form B of claim 1, comprising:
dissolving quinine sulfate in ethanol to form a solution and evaporating the ethanol until quinine Form B forms; or
dissolving quinine sulfate in ethanol at about 31 to about 78° C. to form a solution, and
cooling the solution.

4. Form C quinine sulfate of claim 1, comprising:
XRPD peak positions at 6.2, 9.2, 12.9, 14.0. 15.3, 16.6, 17.5, and 18.4 ±0.2 degrees 2-theta;
peaks according to Table 2 ±0.2 degrees 2-theta; or
an X-ray powder diffraction pattern which is substantially similar to FIG. 2.

5. A process for preparing quinine sulfate Form C of claim 1, comprising:
dissolving quinine sulfate in ethanol to form a solution and adding an aliphatic hydrocarbon antisolvent until quinine Form C forms.

6. Form D quinine sulfate of claim 1, comprising:
XRPD peak positions at 8.6, 9.7, 14.1, 16.8, 18.1, 19.9, and 21.3 ±0.2 degrees 2-theta;
peaks according to Table 3 ±0.2 degrees 2-theta; or
exhibiting an X-ray powder diffraction pattern which is substantially similar to FIG. 3,
wherein quinine sulfate Form D is substantially free of quinine sulfate Form A.

7. A process for preparing quinine sulfate Form D of claim 1, comprising:
vacuum drying quinine sulfate Form A at about 55 to about 65° C. for about 11 days or more to form quinine sulfate Form D.

8. Form E quinine sulfate of claim 1, comprising:
XRPD peak positions at 8.3, 14.4, 16.2, 17.9, 18.8, 22.4 and 26.0 ±0.2 degrees 2-theta;
peaks according to Table 4 ±0.2 degrees 2-theta; or
exhibiting an X-ray powder diffraction pattern which is substantially similar to FIG. 4.

9. A process for preparing quinine sulfate Form E of claim 1, comprising:
dissolving quinine sulfate in methanol to form a solution, and
adding an aqueous antisolvent until quinine sulfate Form E forms; or
dissolving quinine sulfate in 2,2,2-trifluoroethanol and water with heating to form a solution and allowing the solution to cool until quinine sulfate Form E forms.

10. Form F quinine sulfate of claim 1, comprising:
XRPD peak positions at 7.5, 8.3, 15.4, 17.5, and 20.6 ±0.2 degrees 2-theta;
peaks according to Table 5 ±0.2 degrees 2-theta; or
exhibiting an X-ray powder diffraction pattern which is substantially similar to FIG. 5.

11. A process for preparing quinine sulfate Form F of claim 1, comprising:
slurrying quinine sulfate in ethanol for 5 days or greater, wherein the slurrying is carried out at a temperature of about 40 to about 60° C., and
optionally the ethanol is allowed to evaporate during the slurrying process.

12. Form G quinine sulfate of claim 1, comprising:
XRPD peak positions at 6.1, 7.7, 16.8, 17.9, and 18.8 ±0.2 degrees 2-theta;
peaks according to Table 6 ±0.2 degrees 2-theta; or
exhibiting an X-ray powder diffraction pattern which is substantially similar to FIG. 6.

13. A process for preparing quinine sulfate Form G of claim 1, comprising:
slurrying quinine sulfate in tert-butanol for 3 days or greater, wherein the slurrying is carried out at a temperature of about 40 to about 60° C.

14. Form H quinine sulfate of claim 1, comprising:
XRPD peak positions at 7.9, 9.1, 13.9, 15.8, 16.5, 17.2, 17.8, and 18.1 ±0.2 degrees 2-theta;
peaks according to Table 7 ±0.2 degrees 2-theta; or
exhibiting an X-ray powder diffraction pattern which is substantially similar to FIG. 7.

15. A process for preparing quinine sulfate Form H of claim 1, comprising:
dissolving quinine sulfate in hexafluoroisopropanol and allowing the solvent to evaporate until quinine sulfate Form H forms.

16. Form I quinine sulfate of claim 1, comprising:
XRPD peak positions at 8.3, 10.5, 14.8, 15.6, 17.2, 17.6, 18.9, 19.2, and 20.9 ±0.2 degrees 2-theta;
peaks according to Table 8 ±0.2 degrees 2-theta; or
exhibiting an X-ray powder diffraction pattern which is substantially similar to FIG. 8.

17. A process for preparing quinine sulfate Form I of claim 1, comprising:
slurrying quinine sulfate in methanol for 5 days or greater, wherein the slurrying is carried out at a temperature of about 40 to about 60° C., and
optionally the methanol is allowed to evaporate during the slurrying process.

18. Form J quinine sulfate of claim 1, comprising:
XRPD peak positions at 4.2, 6.1, 15.8, 18.4, 20.4, 21.4, and 23.6 ±0.2 degrees 2-theta;
peaks according to Table 9 ±0.2 degrees 2-theta; or
exhibiting an X-ray powder diffraction pattern which is substantially similar to FIG. 9.

19. A process for preparing quinine sulfate Form J of claim 1, comprising:
suspending quinine sulfate in a combination of 1,1,1-trifluoroethanol and tetrahydrofuran to form a mixture;
sonicating the mixture; and
allowing the 1,1,1-trifluoroethanol and tetrahydrofuran to evaporate under ambient conditions or under reduced pressure.

20. Form K quinine sulfate of claim 1, comprising:
XRPD peak positions at 4.3, 6.1, 6.4, 8.9, 17.7, 19.1, and 23.3 ±0.2 degrees 2-theta;
peaks according to Table 10 ±0.2 degrees 2-theta; or
exhibiting an X-ray powder diffraction pattern which is substantially similar to FIG. 10.

21. A process for preparing quinine sulfate Form K of claim 1, comprising:
slurrying quinine sulfate in tetrahydrofuran for 8 days or greater, wherein the slurrying is carried out at a temperature of about 40 to about 60° C.

22. Quinine sulfate Form A, B, C, D, E, F, G, H, I, J, or K of claim 1 substantially free of other quinine sulfate polymorphs.

23. Quinine sulfate Form A, B, C, D, E, F, G, H, I, J, or K of claim 1 having a purity of greater than or equal to about 95, 96, 97, 98, 99 or 99.5%.

24. Non-crystalline quinine sulfate of claim 1 exhibiting an X-ray powder diffraction pattern is substantially free of peaks of crystalline quinine sulfate forms.

25. A composition comprising:
quinine sulfate Form A, B, C, D, E, F, G, H, I, J, K or a noncrystalline form; and
a pharmaceutically acceptable excipient.

26. A method of treating a patient, comprising:
administering to a patient in need thereof quinine sulfate Form A, B, C, D, E, F, G, H, I, J, K; a noncrystalline form; or a combination thereof.

27. A method of treating a patient, comprising:
administering to a patient in need thereof the composition of claim 25.

28. The method of claim 27, wherein the composition is used to treat malaria caused by Plasmodium species, uncomplicated Plasmodium falciparum malaria, severe or complicated Plasmodium falciparum malaria, malaria caused by Plasmodium vivax, leg muscle cramps, or babesiosis; or for the prophylaxis of malaria or leg muscle cramps.
